(19) United States
(12) Patent Application Publication
(10) Pub. No.: US 2020/0155000 A1
(43) Pub. Date: May 21, 2020

(54) FETAL MOVEMENT MEASURING DEVICE

(71) Applicant: SOUTHERN TAIWAN UNIVERSITY OF SCIENCE AND TECHNOLOGY, Tainan City (TW)

(72) Inventors: Yi Chun Du, Tainan City (TW); Bee Yen Lim, Tainan City (TW)

(21) Appl. No.: 16/718,041

(22) Filed: Dec. 17, 2019

Related U.S. Application Data
(63) Continuation-in-part of application No. 15/460,453, filed on Mar. 16, 2017.

(30) Foreign Application Priority Data
Mar. 17, 2016 (TW) 105108296

(51) Int. Cl. A61B 5/00; A61B 5/0205; A61B 5/11; A61B 5/0448

(52) U.S. Cl. CPC A61B 5/0011; A61B 5/0205; A61B 5/1118; A61B 2562/0219; A61B 5/0022; A61B 5/4362; A61B 5/0448

(57) ABSTRACT

A fetal movement measuring device includes a detection unit and a smart device. The detection unit is selected from elastic materials and is attachable to an abdomen of a pregnant woman. The detection unit includes multiple branch sections extending outward therefrom. Each of the branch sections includes a fetal movement sensor and an electrode patch. The fetal movement sensor and the electrode patch are connected to a main circuit board to transmit an abdominal dynamic physiological signal detected by the fetal movement sensor and a fetal electrocardiographic signal detected by the electrode patch to the main circuit board. The smart device is operable to receive the abdominal dynamic physiological signal and the fetal electrocardiographic signal from the main circuit board and includes a fetal movement algorithm program that calculates and generates fetal movement data and the fetal heart rate.

### CROSS REFERENCE TO RELATED APPLICATIONS

This is a continuation-in-part application of co-pending U.S. patent application Ser. No. 15/460,453, “Fetal Movement Measuring Device”, filed on Mar. 16, 2017.

### TECHNICAL FIELD OF THE INVENTION

The present invention relates to a measuring device, and more particularly to a fetal movement measuring device.

### DESCRIPTION OF THE PRIOR ART

Fetal movement, uterine contraction, and fetal heart rate are three major physiological parameters by which the condition of a fetus can be known during pregnancy of the mother. Fetal movement refers to the movement of a fetus in the uterus. Uterine contraction refers particularly to the pressure generated by such contraction. Fetal heart rate refers to the speed of the fetus's heartbeats. It is necessary for a pregnant mother to constantly measure fetal movement in order to ensure safety of the fetus.

Based on the result of clinical interviews and feedback from users, it is commonly known that prenatal care examination equipment that has been currently used can be further improved and does not completely suit the need of clinical uses. For example, ultrasonic equipment is generally hard to get minimized and consumes a large amount of electrical power, and also causes certain safety concerns for long-term use, and lacks detection techniques for accurately measuring site and force of fetal movement in clinical uses.

Further, preterm birth or preemie is a situation that many countries worldwide have to face immediately. Under the conditions that the birth rate is dropping while the rate of premature delivery is increasing, it becomes even further important in today's society to provide an excellent pregnant women caring system in order to protect the safety of pregnant women and fetuses and also to reduce the rate of premature delivery

Thus, it is quite an issue to allow a pregnant woman to do measurement all by herself without being interfered with by the surroundings and to reduce the frequency that the pregnant woman has to travel to hospitals.

### SUMMARY OF THE INVENTION

An objective of the present invention is to provide a fetal movement measuring device, which includes functions of intelligent monitoring and instant analysis of fetal movement, fetal heart rate, and uterine contraction of a pregnant woman by changing techniques based on normal force and Doppler ultrasonography that are adopted in the known technology, in order to establish a detection model of uterine contraction, site of fetal movement, and relative force according to a fetal movement sensor (such as a an inertial sensor) and to establish a fetal heart rate measurement technique based on a non-ultrasonic configuration by applying a physiological multi-channel electrode patch measuring model.

To achieve the above objective, the present invention comprises a detection unit and a smart device, wherein the detection unit is selected from elastic materials and is adapted to attach to an abdomen, the detection unit comprising multiple branch sections extending outward therefrom, each of the branch sections comprising a fetal movement sensor and an electrode patch, wherein the fetal movement sensor and the electrode patch are connected to a main circuit board to transmit an abdominal dynamic physiological signal detected by the fetal movement sensor and a fetal electrocardiographic signal detected by the electrode patch to the main circuit board; and the smart device is operable to receive the abdominal dynamic physiological signal and the fetal electrocardiographic signal from the main circuit board and comprises a fetal movement algorithm program that calculates and generates fetal movement data and the fetal heart rate.

The fetal movement sensors and the electrode patches are arranged in a multi-channel configuration to detect uterine contraction, fetal movement, and fetal heart rate of a pregnant woman and to combine these data. The detection unit is readily deformable or shape-changeable for tight attachment to the surface of the skin of the pregnant woman to enhance comfortableness of wearing, allowing for long-term wearing, and also enhancing easiness of fabrication, to thereby allow the pregnant woman to carry out measurements by herself without being interfered with by the surroundings and reduce the frequency that the pregnant woman has to travel to hospitals for examinations.

### BRIEF DESCRIPTION OF THE DRAWINGS

FIG. 1 is a perspective view of the present invention.

FIG. 2 is a perspective view of the present invention taken from a different angle.

FIG. 3 is a schematic view showing a way of use of the present invention in an assembled form.

FIG. 4 is a perspective view showing an example of another structure of the present invention.

FIG. 5 is a perspective view showing an example of a further structure of the present invention.

FIG. 6 is a schematic view showing another way of use of the present invention.

### DETAILED DESCRIPTION OF THE PREFERRED EMBODIMENT

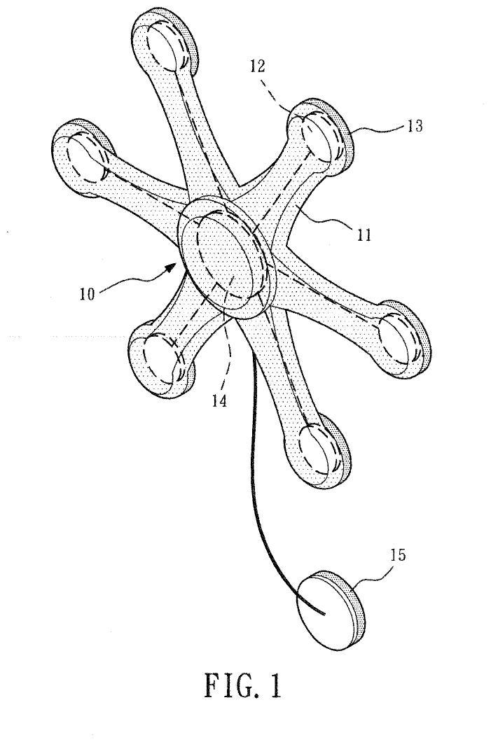

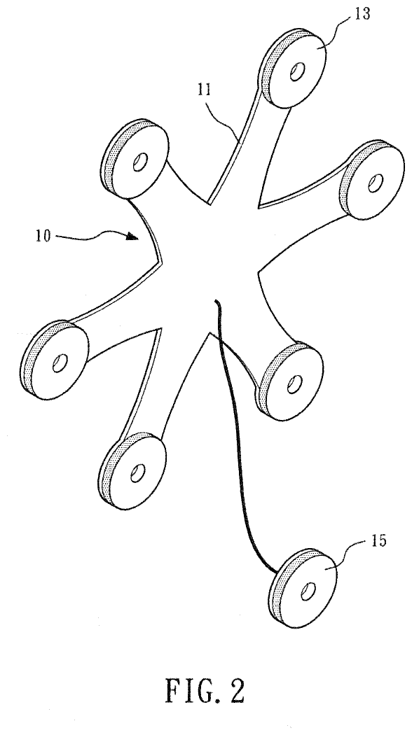

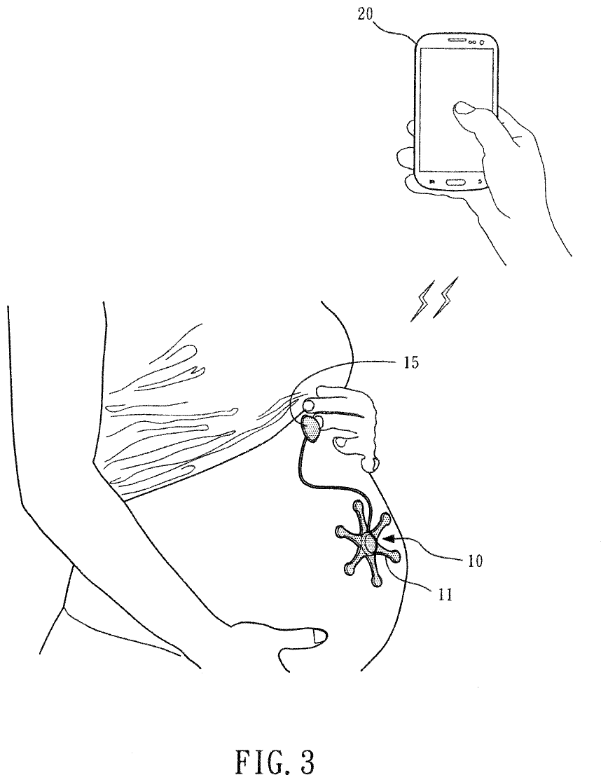

Referring to FIGS. 1-3, the present invention comprises a detection unit 10 and a smart device 20. Details are provided below:

The detection unit 10 is selected from elastic materials and is structured for attaching to an abdomen. The detection unit 10 is extended outward to form multiple branch sections 11. Each of the branch sections 11 includes a fetal movement sensor 12 and an electrode patch 13. The fetal movement sensor 12 and the electrode patch 13 are connected to a main circuit board 14 to transmit an abdominal dynamic physiological signal detected by the fetal movement sensor 12 and a fetal electrocardiographic signal detected by the electrode patch 13 to the main circuit board 14.

The smart device 20 is operable to acquire or otherwise receive the abdominal dynamic physiological signal and the fetal electrocardiographic signal from the main circuit board 14 and is operable with algorithm programs for fetal movement and fetal heart rate to compute and generate fetal movement data and fetal heart rate.

In an embodiment, the fetal movement sensor 12 comprises an inertial measurement unit (IMU).

In an embodiment, the electrode patch 13 includes an adhesive surface, which is releasably attachable to and thus fixed to the abdomen.

In an embodiment, the smart device 20 comprises one of a mobile phone and a computer, which is operable to instantly display the fetal movement data and the fetal heart rate.

The above provides a description to components of the present invention and assembly thereof, and in the following, the features and advantages of use of the present invention will be described.

The fetal movement sensors 12 and the electrode patches 13 that are arranged in a multi-channel configuration are used to detect uterine contraction, fetal movement, and fetal heart rate of a pregnant woman and to combine these data together, wherein the fetal movement sensor 12 detects uterine contraction and fetal movement of the pregnant woman and the electrode patch 13 detects the fetal heart rate inside the pregnant woman.

The detection unit 10 is readily deformable or shape-changeable to tightly attach to the surface of the skin of a pregnant woman to help improve comfortableness of wearing for long-term wearing and also help eases fabrication and assembly thereof, so that the pregnant woman is allowed to be measurement by herself without being interfered with by the surroundings and the frequency that the pregnant woman has to travel to hospitals for examination can be reduced.

In an embodiment, since it is essential for a pregnant woman to stay in a still condition in order to carry out accurate measurement in counting fetal movement, the fetal movement algorithm program comprises an asynchronous signal analysis algorithm program to separate signals of fetal movement, uterine contraction, and daily oscillation/shaking, so that the pregnant woman may immediately get aware of any occurrence of fetal movement or uterine contraction by means of a smart device, such as a mobile phone, even when she is doing housework or walking.

Further, clinical judgement rules and grey relational analysis (GRA) may also be included as being integrated in the present invention to establish a related instantaneous analysis system based on the class determination criterion of fetal heart rate and uterine contraction defined by the National Institute of Child Health and Human Development (NICHD) in order to provide references to clinical personnel in doing examination and caring for pregnant women.

In an embodiment, the main circuit board 14 is connected to a reference electrode patch 15 to detect an electrocardiographic signal of the pregnant woman.

As such, the multiple channels of the electrode patches 13 and the reference electrode patch 15 are operable to detect related electrocardiographic signals and an algorithm program may be provided to separate the heart beats of the pregnant woman and the fetus, in order to acquire actual fetal heart beat and to calculate and determine the fetal heart rate.

In an embodiment, the smart device 20 is operable to transmit the fetal movement data so measured and acquired, through a network, to a cloud server to allow a medical monitoring facility to download the fetal movement data from the cloud server.

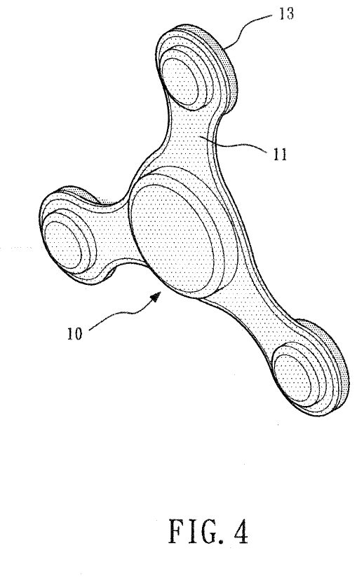

Referring to FIG. 4, the detection unit 10 may alternatively include three such branch sections 11.

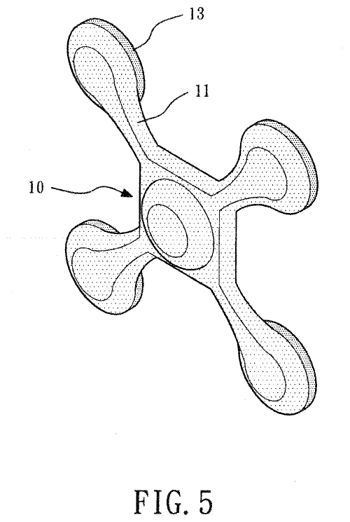

Referring to FIG. 5, the detection unit 10 may alternatively include four such branch sections 11.

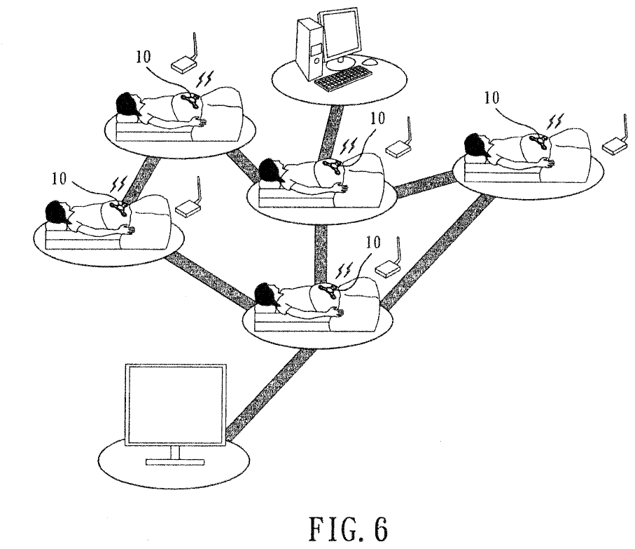

Referring to FIG. 6, the present invention may be used as an application to a pregnant woman examination facility, such as a hospital, to provide a multi-bed monitoring and control configuration for pregnant woman examination.

The present invention provides at least the following features:

(1) Multiple inertial measurement units are used in combination for disposition and attaching to the skin of abdomen of a pregnant woman and an asynchronous signal analysis algorithm program is involved to carry out, in a non-invading manner, detection of site and magnitude of fetal movement.

(2) A multi-channel electrode patch configuration is provided to detect electrocardiographic signals and algorithm programs are applied to separate heart beats of a pregnant woman and a fetus in order to acquire actual fetal heart beat and calculate and determine fetal heart rate.

(3) An arrangement of related circuits and devices is involved to greatly reduce interference of the surroundings and influences caused by oscillation/shaking of the devices to meet the needs for a wearable device.

(4) Detected data can be transmitted, as desired, to a cloud database of a hospital to serve as an important basis for assessment required in subsequent pregnant woman examinations.

(5) Clinical medical personnel is allowed to get immediate aware of the current situation of a pregnant woman by using the cloud database of a pregnant woman examination facility.

(6) The present invention has a simple operation of assembling and a non-professional person may readily wear and use.

I claim:

1. A fetal movement measuring device, comprising:
a detection unit, which is selected from elastic materials and is adapted to attach to an abdomen, the detection unit comprising multiple branch sections extending outward therefrom, each of the branch sections comprising a fetal movement sensor and an electrode patch, wherein the fetal movement sensor and the electrode patch are connected to a main circuit board to transmit an abdominal dynamic physiological signal detected by the fetal movement sensor and a fetal electrocardiographic signal detected by the electrode patch to the main circuit board; and
a smart device, which is operable to receive the abdominal dynamic physiological signal and the fetal electrocardiographic signal from the main circuit board and comprises a fetal movement algorithm program that calculates and generates fetal movement data and the fetal heart rate.

2. The fetal movement measuring device according to claim 1, wherein the fetal movement sensor 12 comprises an inertial measurement unit (IMU).

3. The fetal movement measuring device according to claim 1, wherein the electrode patch comprises an adhesive surface adapted to releasably attach to and thus fix to the abdomen.

4. The fetal movement measuring device according to claim 1, wherein the smart device comprises one of a mobile phone and a computer, which is operable to instantaneously display the fetal movement data and the fetal heart rate.

5. The fetal movement measuring device according to claim 1, wherein the fetal movement algorithm program comprises an asynchronous signal analysis algorithm program, which separates signals of fetal movement, uterine contraction, and a daily oscillation/shaking.

6. The fetal movement measuring device according to claim 1, wherein the main circuit board is connected to a reference electrode patch, which detects an electrocardiographic signal of a pregnant woman.

7. The fetal movement measuring device according to claim 1, wherein the smart device is operable to transmit the fetal movement data through a network to a cloud server, so that a medical monitoring facility is allowed to download the fetal movement data from the cloud server.
